(19) United States
(12) Patent Application Publication
(10) Pub. No.: US 2004/0086547 A1
(43) Pub. Date: May 6, 2004

(54) Compositions for reducing hypercholesterolemia and controlling of postprandial blood glucose and insulin levels

(75) Inventors: Robert Lawrence Prosise, Cincinnati, OH (US); Kevin Patrick Christmas, Mason, OH (US); Joseph James Elsen, St. Bernard, OH (US); Ralph Lawrence Helmers JR., Cincinnati, OH (US); Jeffrey John Kester, West Chester, OH (US); Thomas Joseph Wehmejer, Cincinnati, OH (US)

Correspondence Address: MICHAEL BEST & FRIEDRICH, LLP, 100 E WISCONSIN AVENUE, MILWAUKEE, WI, 53202, US

(73) Assignee: Mid-America Commercialization Corporation

(21) Appl. No.: 10/689,764

(22) Filed: Oct. 21, 2003

Related U.S. Application Data
(62) Division of application No. 09/827,834, filed on Apr. 6, 2001, now abandoned.
(60) Provisional application No. 60/196,410, filed on Apr. 12, 2000.

(51) Int. Cl. A61K 31/715; A61K 31/225; A61K 9/68

(52) U.S. Cl. 424/440; 514/54; 514/547

(57) ABSTRACT

Beta-glucan soluble fiber and non-digestible fats are administered orally to reduce blood cholesterol levels and to control postprandial blood glucose and insulin levels. The beta-glucan soluble fiber and non-digestible fat may be administered as separate compounds, as a mixture, or combined with other materials and administered in the form of an appealing food.

### CROSS REFERENCE TO RELATED APPLICATIONS

This application claims the benefit of U.S. Provisional Application Serial No. 60/196,410 filed on Apr. 12, 2000, in the name of Prosise et al.

### FIELD OF THE INVENTION

The present invention relates to methods and compositions for reducing blood cholesterol levels, and for controlling of postprandial blood glucose and insulin levels, by oral administration of beta-glucan soluble fiber and a non-digestible fat.

### BACKGROUND OF THE INVENTION

High blood cholesterol (hypercholesterolemia) is recognized as a risk factor for coronary heart disease, which is a major health care problem. Epidemiological studies have demonstrated that, with few exceptions, populations consuming large quantities of saturated fat and cholesterol have a relatively high concentration of serum cholesterol and a high mortality rate from coronary heart disease. While it is recognized that other factors can also contribute to the development of cardiovascular disease, there appears to be a causal relationship between the concentration of serum cholesterol, in which hypercholesterolemia results in the accumulation of undesirable amounts of cholesterol in various parts of the circulatory system (arteriosclerosis) or in soft tissues (xanthomatosis), and coronary disease and coronary mortality rates. It is well accepted that lowering of blood cholesterol levels will reduce the risk of heart disease, as well as slow the progression of this chronic disease in individuals already suffering its effects. Typical therapy for persons with hypercholesterolemia includes strict control of dietary intake of fat, saturated fat, and cholesterol. In certain cases, this dietary regimen may be combined with a treatment of cholesterol lowering drugs, such as the bile acid sequestrants (e.g., colestipol and cholestyramine). Patient adherence to a stringent and prolonged dietary and drug regimen is often poor. The cholesterol lowering drugs can have unpleasant side effects and palatability is often extremely poor. Therefore, it would be particularly advantageous to have palatable food products that can replace or enhance the effectiveness of cholesterol lowering drugs.

Diabetes mellitus is a chronic disease, affecting more than 16 million Americans that results from an impairment in the body's ability to produce and/or utilize insulin. Insulin is the hormone necessary for the uptake of glucose into cells where it is used for energy. Type 2 diabetes is the specific form of this disease characterized by insulin resistance, i.e., the body does not efficiently utilize the insulin that is produced. The incidence of Type 2 diabetes is increasing in the United States and other western societies, and this has been associated with the prevalence of obesity and the lack of physical activity. Uncontrolled diabetes can lead to high blood levels of glucose and insulin, which ultimately can result in damage to various organs and blood vessels. Treatment for diabetes tends to focus on normalizing blood glucose levels, as well as promoting weight loss if needed. Relatively low-fat and low-calorie, palatable, food compositions that help to control postprandial blood glucose levels would be a convenient and effective dietary option for diabetic individuals.

In the present invention, non-digestible fats are used in combination with beta-glucan soluble fiber as orally-administered compositions for the primary objective of reducing blood cholesterol levels, and for a secondary objective of controlling postprandial blood glucose and insulin levels. In one mode, the compositions herein are combined with other materials to form embodiments that are as appealing as many snack foods.

### SUMMARY OF THE INVENTION

The present invention encompasses orally-administered compositions of matter, for the primary objective of reducing blood cholesterol levels and the secondary objective of controlling postprandial blood glucose and insulin levels in humans or lower animals, comprising a mixture of:

(a) beta-glucan soluble fiber or a source of beta-glucan soluble fiber; and

(b) a non-digestible fat or a source of non-digestible fat.

The invention also encompasses a method for reducing blood cholesterol levels and controlling postprandial blood glucose and insulin levels in a patient (including both humans and lower animals) in need of such treatment, comprising orally administering to said patient a safe and effective amount of:

(a) beta-glucan soluble fiber or a source of beta-glucan soluble fiber; and

(b) a non-digestible fat or a source of non-digestible fat.

The compositions herein can be provided in bulk form as granules, or in unit dosage forms such as tablets, capsules, effervescing granules or tablets, and the like. The compositions can contain various flavorings, extenders, tableting aids, and the like, well-known to formulators of pharmaceutical products.

In an optional embodiment, the compositions herein can be in the form of appealing foods, including traditional snack foods.

### Definitions

As used herein, the term “traditional snack” means: 1) baked goods selected from the group consisting of crackers, cookies, brownies, filled crackers, snack cakes, pies, granola bars, and toaster pastries; 2) salted snacks selected from the group consisting of potato crisps, corn chips, tortilla chips, extruded snacks, filled extruded snacks, enrobed extruded snacks and pretzels; 3) specialty snacks selected from the group consisting of dips, spreads, meat snacks and rice/corn cakes; and 4) confectionary snacks.

As used herein, the term “fat” refers to the total amount of digestible, partially digestible and non-digestible fats or oils that are present in the embodiments of the present invention. For purposes of this invention, emulsifiers are considered to be fats.

As used herein, the terms “lipid”, “fat” and “oil” are synonymous.

As used herein, the term “non-digestible fat” refers to the total amount of non-digestible fats or oils that are present in the embodiments of the present invention.

As used herein, the term “carbohydrate” refers to the total amount of sugar alcohols, monosaccharides, disaccharides, oligosaccharides, digestible, partially digestible and non-digestible polysaccharides; and lignin or lignin like materials that are present in the embodiments of the present invention.

As used herein, the term “ready-to-eat” when used to describe Applicants' invention, means that after manufacture and packaging, Applicants' invention requires no additional processing, including but not limited to cooking, baking, microwaving, boiling, frying; or combination with components outside of the product's packaging. However, this does not rule out that any of the parameters of Applicants' invention, for example, the invention's appeal or taste, may be improved when said compositions are processed further or combined with other foods.

As used herein, the term “single serving” means any quantity of food sold, marked, described, advertised, or implied to be equivalent to a single serving size or unit. For example, in the U.S., single serving sizes for foods are defined in the FDA Labeling Rules as contained in 21 CFR § 101.12 which is incorporated herein by reference in its entirety.

As used herein, the articles a and an when used in a claim, for example, “a fat” is understood to mean one or more of the material that is claimed or described.

Publications, patents, and patent applications are referred to throughout this disclosure. All references cited herein are hereby incorporated by reference.

Unless indicated otherwise, all percentages and ratios are calculated by weight.

Unless indicated otherwise, all percentages and ratios are calculated based on the total composition.

Unless otherwise noted, all component or composition levels are in reference to the active level of that component or composition, and are exclusive of impurities, for example, residual solvents or byproducts, which may be present in commercially available sources.

### DETAILED DESCRIPTION OF THE INVENTION

A variety of dietary and drug regimens have been suggested for alleviating or preventing hypercholesterolemia and for controlling postprandial blood glucose and insulin levels. It is well known that many of these regimes have undesirable side effects, give suboptimal results, or require the consumption of numerous unappealing substances and a significant reduction in the consumption of appealing foods. It is also well known that many consumers, particularly those individuals most in need of treatment, prefer appealing fat and sugar laden foods to foods required by their prescribed dietary regimes. Since many individuals associate the form of a food with the enjoyment of the eating experience, individuals are more likely to follow their prescribed dietary regime, and thereby obtain the benefits of treatment, when the prescribed food is similar, at least in form, to an appealing but unhealthy food. As a result, what is needed, in addition to compositions comprising beta-glucan soluble fiber and a non-digestible fat, is one or more foods comprising beta-glucan soluble fiber and a non-digestible fat, wherein the food has the form, and preferably the taste and texture, of an appealing but unhealthy food.

Unfortunately, the incorporation of various β-glucan containing ingredients into food products results in challenging taste and/or process performance issues. For example, Jenkins et al. (2001, in Advanced Dietary Fiber Technology, B. V. McCleary and L. Prosky, ed., Blackwell Science Ltd., pg. 162-167) highlight the point that, while viscous fibers (e.g. β-glucan) have indeed shown promise for the treatment of diabetes, “the lack of readily available palatable formulations has made further work in this area difficult”. Also, it is known that the incorporation of whole grain sources of β-glucan soluble fiber (e.g. oat bran) into yeast-leavened baked goods can stress the gluten protein matrix, thereby resulting in a reduced loaf volume and a more dense texture (Stauffer, C. E., 2000, Baking & Snack, February issue, pg. 64-70). For certain baked goods, such as muffins, expansion and development of the desired texture during baking is dependent upon the proper batter viscosity. Unfortunately, viscous fibers, such as β-glucan soluble fiber, can absorb a significant quantity of water, which increases batter viscosities, thus resulting in products with poor textures. Finally, the incorporation of whole grain sources of β-glucan soluble fiber (e.g. oat bran) into low-moisture baked or fried foods (e.g. crackers, cookies, crisps) may result in excessive tenderizing of the structure of the product.

Beta-Glucan Soluble Fiber

Beta-glucan soluble fiber is a high molecular weight polysaccharide composed of β-(1→4)-linked glucose units, separated every 2-3 units by a single β-(1→3)-linked glucose unit. Beta-glucan soluble fiber occurs in all cereal grains, with contents ranging from less than 2% to in excess of 6% by weight (Glicksman, M., October 1991, Food Technology, pg. 94).

A slightly different form of β-glucan soluble fiber can also be isolated from baker's or brewer's yeast; e.g., Saccharomyces cerevisiae (Jamas, S. et al., 1990, U.S. Pat. No. 4,962,094; Sucher, R. W. et al., 1975, U.S. Pat. No. 3,867,554). This polysaccharide is a (1→6), (1→3)-β-D-glucan, composed of β-(1→3)-linked glucose units with β-(1→6)-linked glucose branches.

Consumption of β-glucan soluble fiber provides physiological benefits that include lowering of blood serum total and low density lipoprotein (LDL) cholesterol in hypercholesterolemic subjects, as well as attenuation of the postprandial rise in blood glucose and insulin levels (Kahlon, T. S., 2001, in Advanced Dietary Fiber Technology, B. V. McCleary and L. Prosky, ed., Blackwell Science Ltd., pg. 206-220; Wood, P. J., 2001, in Advanced Dietary Fiber Technology, B. V. McCleary and L. Prosky, ed., Blackwell Science Ltd., pg. 315-327; Bratten, J. T. et al., 1994, Diabetes Medicine, 11:312-318; Tappy, L. et al., 1996, Diabetes Care, 19:831-834; Bratten, J. T. et al., 1994, Eur. J. Clin. Nutr., 48:465-474). While not intending to be limited by theory, it is believed that the mechanism by which β-glucan soluble fiber helps lower blood cholesterol levels is related to thickening or gelation of the intestinal contents, which reduces absorption of dietary cholesterol and re-absorption of bile acids. The increase in bile acid excretion results in a reduced bile acid pool circulating back to the liver. This, in turn, causes the body to compensate by synthesizing additional bile acids from the endogenous cholesterol stores, which results in a lowering of the plasma cholesterol level. In addition, β-glucan soluble fiber is at least partially fermented by the microflora in the large bowel to produce the short chain fatty acids, acetate, butyrate, and propionate. The short chain fatty acids are absorbed and transported to the liver, where they exert an inhibitory effect on hepatic cholesterol synthesis (Bell, S. et al. 1999, Crit. Rev. Food Sci. Nutr., 39 (2): 189-202). The benefits that β-glucan soluble fiber provides for control of postprandial blood glucose and insulin levels are also believed to be due to increased viscosity of the contents in the small intestine, which delays gastric emptying and slows the rate of absorption of glucose and other nutrients. The slower rate of nutrient absorption results in a lower level of insulin secretion required for the cellular uptake of glucose. Hence, postprandial insulin levels are reduced. The lower rise in blood glucose and insulin that results is beneficial to both healthy and diabetic individuals.

Sources of beta-glucan soluble fiber that are useful in practicing Applicants' invention include but are not limited to cereal grains such as oats, barley, rye and yeast-derived β-glucan isolates. Specific examples of useful oat derived sources include but are not limited to Quaker Oats oat bran, Quaker Oats Oatcor® brand of oat bran concentrate, and Oatrim®, a enzymatically hydrolyzed oat flour, all of which are supplied by The Quaker Oats Company and the Beta-Trim™ brand of Quaker Oatrim® available from Rhone-Poulenc Food Ingredients (Cranbury, N.J.). Additional useful oat derived sources of beta-glucan soluble fiber include High β-Glucan Oatrim preparations supplied by Rhone-Poulenc Food Ingredients (Cranbury, N.J.) and oat gum, a β-glucan enriched isolate derived from oats. Oat gum preparations containing 70-90% β-glucan have been described in the literature (Wood, P. J. et al., 1989, Cereal Chem. 66 (2):97-101; Doublier, J. and Wood, P. J., 1995, Cereal Chem. 72 (4):335-340).

Useful barley derived sources of β-glucan include but are not limited to whole barley, as well as barley flakes, flour, and bran.

Useful rye derived sources of β-glucan include but are not limited to rye flour and rye bran.

Useful yeast-derived β-glucan isolates include but are not limited to Fibercel® that is supplied by Alpha-Beta Technology, Inc., of Worcester, Mass.

The concentrations of beta-glucan soluble fiber in Applicants' examples of useful beta-glucan soluble fiber sources are provided by the supplier of the material or can be obtained by analyzing said materials according to Applicants' analytical method for determining beta-glucan soluble fiber levels.

Each embodiment of Applicants' invention contains at least about 0.5 gram of beta-glucan soluble fiber per single serving of an embodiment. Other embodiments of Applicants' invention contain at least about 0.75 grams of beta-glucan soluble fiber per single serving of each embodiment. Still other embodiments of Applicants' invention contain at least about 1.0 gram of beta-glucan soluble fiber per single serving of an embodiment. Still other embodiments of Applicants' invention contain at least about 2.5 grams of beta-glucan soluble fiber per single serving of an embodiment. Still other embodiments of Applicants' invention contain from about 0.5 gram to about 7.5 grams of beta-glucan soluble fiber per single serving of an embodiment.

Other embodiments of Applicants' invention contain at least about 0.5 gram of beta-glucan soluble fiber per 30 grams of embodiment. Other embodiments of Applicants' invention contain at least about 0.75 grams of beta-glucan soluble fiber per 30 grams of embodiment. Still other embodiments of Applicants' invention contain at least about 1.0 gram of beta-glucan soluble fiber per 30 grams of embodiment. Still other embodiments of Applicants' invention contain at least about 2.5 grams of beta-glucan soluble fiber per 30 grams of embodiment. Still other embodiments of Applicants' invention contain from about 0.5 gram to about 7.5 grams of beta-glucan soluble fiber per 30 grams of embodiment.

Doughs used to produce certain embodiments of Applicants' invention contain at least about 1% beta-glucan soluble fiber by weight. Other doughs used to produce certain embodiments of Applicants' invention contain at least about 2% beta-glucan soluble fiber by weight. Still other doughs used to produce certain embodiments of Applicants' invention contain from about 1% to about 10% beta-glucan soluble fiber by weight. Surprisingly, certain embodiments of the above mentioned doughs are found to be sheetable.

Non-Digestible Fat

A key component of Applicants' compositions is a non-digestible fat. Suitable non-digestible edible lipids for use herein include polyol fatty acid polyesters (see Jandacek; U.S. Pat. No. 4,005,195; Issued Jan. 25, 1977); esters of tricarballylic acids (see Hamm; U.S. Pat. No. 4,508,746; Issued Apr. 2, 1985); diesters of dicarboxylic acids such as derivatives of malonic and succinic acid (see Fulcher, U.S. Pat. No. 4,582,927; Issued Apr. 15, 1986); triglycerides of alpha-branched chain carboxylic acids (see Whyte; U.S. Pat. No. 3,579,548; Issued May 18, 1971); ethers and ether esters containing the neopentyl moiety (see Minich; U.S. Pat. No. 2,962,419; Issued Nov. 9, 1960); fatty polyethers of polyglycerol (See Hunter et al; U.S. Pat. No. 3,932,532; Issued Jan. 13, 1976); alkyl glycoside fatty acid polyesters (see Meyer et al; U.S. Pat. No. 4,840,815; Issued Jun. 20, 1989); polyesters of two ether linked hydroxypolycarboxylic acids (e.g., citric or isocitric acid) (see Huhn et al; U.S. Pat. No. 4,888,195; Issued Dec. 19, 1988); and esters of epoxide-extended polyols (see White et al; U.S. Pat. No. 4,861,613; Issued Aug. 29, 1989); as well as polydimethyl siloxanes (e.g., Fluid Silicones available from Dow Corning). All of the foregoing patents relating to the non-digestible lipid component are incorporated herein by reference.

Other useful non-digestible lipids include plant sterols and sterol esters. Non-limiting examples of useful plant sterols and sterol esters include sitosterol, sitostanol, campesterol, and mixtures thereof.

Preferred non-digestible lipids are the polyol fatty acid polyesters that comprise sugar polyesters, sugar alcohol polyesters, and mixtures thereof. The preferred sugars and sugar alcohols for preparing these polyol polyesters include erythritol, xylitol, sorbitol, glucose, and sucrose, with sucrose being especially preferred. The sugar or sugar alcohol starting materials for these polyol polyesters are preferably esterified with fatty acids containing from 8 to 22 carbon atoms, and most preferably from 8 to 18 carbon atoms. When sucrose is used to prepare the polyol fatty acid polyesters, the resulting sucrose polyester has on average at least 4, preferably at least 5, fatty acid ester linkages per molecule. Suitable naturally occurring sources of such fatty acids include corn oil fatty acids, cottonseed oil fatty acids, peanut oil fatty acids, soybean oil fatty acids, canola oil fatty acids (i.e. fatty acids derived from low erucic acid rapeseed oil), sunflower seed oil fatty acids, sesame seed oil fatty acids, safflower oil fatty acids, fractionated palm oil fatty acids, palm kernel oil fatty acids, coconut oil fatty acids, tallow fatty acids and lard fatty acids.

Other suitable polyol fatty acid polyesters are esterified linked alkoxylated glycerins, including those comprising polyether glycol linking segments, as described in U.S. Pat. No. 5,374,446, incorporated herein by reference, and those comprising polycarboxylate linking segments, as described in U.S. Pat. Nos. 5,427,815 and 5,516,544, incorporated herein by reference; more preferred are those described in U.S. Pat. No. 5,516,544.

Additional useful polyol fatty acid polyesters are esterified epoxide-extended polyols as described in U.S. Pat. No. 4,861,613 and EP 0324010 A1, incorporated herein by reference. Preferred esterified epoxide-extended polyols include esterified propoxylated glycerols as described in U.S. Pat. Nos. 4,983,329 and 5,175,323, respectively, both incorporated herein by reference. Also preferred are esterified propoxylated glycerols prepared by reacting an epoxide and a triglyceride with an aliphatic polyalcohol, as described in U.S. Pat. No. 5,304,665, incorporated herein by reference, or with an alkali metal or alkaline earth salt of an aliphatic alcohol, as described in U.S. Pat. No. 5,399,728, incorporated herein by reference. More preferred are acylated propylene oxide-extended glycerols as described in U.S. Pat. Nos. 5,603,978 and 5,641,534, both incorporated herein by reference. Particularly preferred are fatty acid-esterified propoxylated glycerols as described in U.S. Pat. Nos. 5,589,217 and 5,597,605, both incorporated herein by reference.

Non-digestible polyol polyesters that are liquid at body temperature are those which have minimal, or no solids at body temperatures (i.e., 98.6° F. 37° C.). These liquid polyol polyesters typically contain ester groups having a high proportion of C12 or lower saturated fatty acid radicals or else a high proportion of C18 or higher unsaturated fatty acid radicals. In the case of those liquid polyol polyesters having high proportions of unsaturated C18 or higher fatty acid radicals, at least about half of the fatty acids incorporated into the polyester molecule are typically unsaturated. Preferred unsaturated fatty acids in such liquid polyol polyesters are oleic acid, linoleic acid, and mixtures thereof.

Polyol fatty acid polyesters that are normally solid at body temperatures can also be useful in the present invention. Particularly preferred solid polyol fatty acid polyesters for use in the present invention are those materials disclosed in U.S. Pat. Nos. 5,306,514; 5,306,515; and 5,306,516, all to Letton et al., all issued Apr. 26, 1994, and all assigned to The Procter & Gamble Company. Said materials are solid polyol polyesters and referred to hereinafter as “high-C20 and above long-chain fatty acid polyol polyesters” and comprise: (1) long chain (at least 12 carbon atoms) unsaturated fatty acid radicals, or a mixture of said radicals and saturated short chain (C2-C12) fatty acid radicals, and (11) long chain (at least 20 carbon atoms) saturated fatty acid radicals, in a molar ratio of 1:11 of from about 1:15 to about 2:1, and wherein at least 4 of the hydroxyl groups of the polyol are esterified.

The polyol fatty acid polyesters suitable for use in the compositions herein can be prepared by a variety of methods known to those skilled in the art. These methods include: transesterification of the polyol (i.e. sugar or sugar alcohol) with methyl, ethyl or glycerol esters containing the desired acid radicals using a variety of catalysts; acylation of the polyol with an acid chloride; acylation of the polyol with an acid anhydride; and acylation of the polyol with the desired acid, per se. (See, for example, U.S. Pat. Nos. 2,831,854, 3,600,186, 3,963,699, 4,517,360 and 4,518,772, all of which are incorporated by reference.

These patents all disclose suitable methods for preparing polyol polyesters.) The most preferred non-digestible fat is olestra (Olean® brand, The Procter & Gamble Co., Cincinnati, Ohio).

Each embodiment of Applicants' invention contains at least about 1 gram of non-digestible fat per single serving of an embodiment. In other embodiments of Applicants' invention, each embodiment contains at least about 4 grams of non-digestible fat per single serving of an embodiment. In still other embodiments of Applicants' invention, each embodiment contains at least about 6 grams of non-digestible fat per single serving of said embodiment. In still other embodiments of Applicants' invention contain from about 1.0 gram to about 16.0 grams of non-digestible fats per single serving of an embodiment.

Other embodiments of Applicants' invention contain at least about 1 gram of non-digestible fat per 30 grams of embodiment. In other embodiments of Applicants' invention, each embodiment contains at least about 4 grams of non-digestible fat per 30 grams of embodiment. In still other embodiments of Applicants' invention, each embodiment contains at least about 6 grams of non-digestible fat per 30 grams of embodiment. In still other embodiments of Applicants' invention contain from about 1.0 gram to about 16.0 grams of non-digestible fats per 30 grams of embodiment.

Water Activities

Applicants' invention is not limited to any particular range of water activity. However, certain embodiments of Applicants' invention have water activities that are less than or equal to 0.90. Other embodiments of Applicants' invention are “non-perishable”, thus they have water activities that are sufficiently low to prevent the growth of most pathogenic and spoilage bacteria; i.e., a water activity less than 0.85 (Troller, J. A. 1980, Influence of Water Activity on Microorganisms in Foods, Food Technology, 34:76-80; Troller, J. A. 1989, Water Activity and Food Quality, in “Water and Food Quality”, T. M. Hardman, ed., pg. 1-31). Other embodiments of Applicants' invention have water activities low enough to control or prevent the growth of yeasts and molds; i.e., a water activity less than 0.80, more preferably less than 0.75, and most preferably less than 0.70.

Adjunct Ingredients

Adjunct ingredients are necessary for processing and structural development of most foods. Examples of typical adjunct ingredients include processing aids, emulsifiers, and leavening agents. As known by those skilled in the art, adjunct ingredients that are needed to produce foods vary by food type. Based on the teachings in Applicants' disclosure and examples, the selection of the appropriate type and level of adjunct is easily determined by one skilled in the art.

Additional Ingredients

Additional ingredients that may be incorporated in Applicants' invention include vitamins, minerals, natural and synthetically prepared flavoring agents, caloric and non-caloric sweeteners, bracers, flavanols, natural and synthetically prepared colors, preservatives, acidulants, and food stability antioxidants.

Embodiments of the present invention may contain vitamins selected from the group consisting of vitamins A, D, E, K, C (ascorbic acid), thiamin, riboflavin, niacin, vitamin B₋₆, folate, vitamin B₋₁₂, biotin, and pantothenic acid. These vitamin sources are preferably present in nutritionally relevant amounts, which means that the vitamin sources used in the practice of this invention provide a nourishing amount of said vitamins. Where necessary for a patient's health or required by regulation, one or more of the fat-soluble vitamins, vitamins A, D, E, and K, can be used to fortify the Applicants' non-digestible fat containing compositions. For example, in the United States products containing non-digestible fats must be fortified with a minimum of 1.9 mg α-tocopherol equivalents (vitamin E) per gram of non-digestible fat; 170 IU of vitamin A per gram of non-digestible fat; 12 IU of vitamin D per gram of non-digestible fat and 81 g of vitamin K per gram of non-digestible fat.

Embodiments of the present invention may be fortified with minerals such as calcium, phosphorus, magnesium, iron, zinc, iodine, selenium, copper, manganese, fluoride, chromium, molybdenum, sodium, potassium, and chloride. The minerals sources are preferably present in nontoxic nutritionally relevant amounts, which means that the mineral sources used in the practice of this invention provide a nourishing amount of said minerals.

If desired, flavoring and/or coloring agents can also be added to the food compositions of the present invention. Any suitable flavoring or coloring agents approved for food use can be utilized for the present invention.

Embodiments of the present invention may contain one or more caloric and/or non-caloric sweetening agents known in the art. Non-caloric, low-calorie, or high-intensity sweetening agents are preferred for certain embodiments because of their favorable contribution to control of postprandial blood glucose levels. Non-limiting examples of sweetening agents include aspartame, neotame, acesulfame K, sucralose, saccharin, polyols such as erythritol, xylitol, mannitol, and sorbitol, and mixtures thereof.

When desired, preservatives, such as sorbic acid, benzoic acid, hexametaphosphate and salts thereof, can be added into embodiments of the present invention.

### METHOD OF TREATMENT

The treatment regimen herein comprises orally administering to a patient, in need of blood cholesterol level reduction or control of postprandial blood glucose and insulin levels, a safe and effective amount of the beta-glucan soluble fiber, or source thereof, and a non-digestible fat of the type described hereinabove, or, conveniently, mixtures of these two materials. To achieve a desired hypocholesterolemic effect (i.e., lowering of blood cholesterol levels) and postprandial blood glucose and insulin control it is important that a patient ingest at least 10 g non-digestible fat and at least 1.5 g beta-glucan soluble fiber per day. Preferably, a sufficient amount of materials are consumed on a daily basis to provide at least 15 g, more preferably at least 20 g, and most preferably from about 25 g to about 40 g non-digestible fat per day; and at least 3 g, more preferably at least 5 g, and most preferably from about 7.5 g to about 15 g beta-glucan soluble fiber per day. Chronic ingestion of beta-glucan soluble fiber and a non-digestible fat is appropriate and preferred in most circumstances. However, appropriate consumption levels and durations can vary with the size and condition of the patient, and the patient's blood cholesterol level. Such matters will, of course, be apparent to the attending physician. Preferably, ingestion of the beta-glucan soluble fiber and non-digestible fat occurs at two, three, or more regularly spaced intervals throughout the day. Again, this can be varied depending on the patient and the attending physician's recommendation.

As mentioned, it is convenient to use the beta-glucan soluble fiber and the non-digestible fat as a mixture. Here, a patient should consume sufficient quantities of said mixture with time to provide the patient with the amounts of beta-glucan soluble fiber and non-digestible fat per time that are recommended above.

A beta-glucan soluble fiber and non-digestible fat mixture is prepared by preparing beta-glucan soluble fiber, or a source of beta-glucan soluble fiber, in a well-known manner, and admixing said fiber with a non-digestible fat, generally in amounts sufficient to produce a composition having a beta-glucan soluble fiber to non-digestible fat weight ratio of from about 1:20 to about 20:1. Other embodiments of Applicants' invention have weight ratios of beta-glucan soluble fiber to non-digestible fat of from about 1:10 to about 10:1. Still other embodiments of Applicants' invention have weight ratios of beta-glucan soluble fiber to non-digestible fat of from about 1:3 to about 3:1. The materials readily admix, particularly when a liquid non-digestible fat, such as sucrose octaoleate, is used; or when a semi-solid non-digestible fat, such as olestra, is heated to a temperature above its melting point (≧140° F.; ≧60° C.) prior to blending.

In one mode, beta-glucan soluble fiber or a source of beta-glucan soluble fiber and a non-digestible fat are combined with other materials to form embodiments that have the form of appealing foods, and that, in many cases, are as appealing in their taste and textural properties as many conventional snack foods. The resulting appealing food compositions are ingested, preferably on a chronic basis, in sufficient amounts and at sufficient intervals to lower serum total and/or LDL cholesterol levels, and/or to control postprandial blood glucose and insulin levels.

### Analytical Protocols

Protocols used to determine the levels and types of beta-glucan soluble fiber and fat, as well as the water activities of embodiments of Applicants' invention, are as follows:

1. Digestible Fat and Digestible Saturated Fat: The content of total digestible fat and digestible saturated fat in a food is measured according to the published AOAC peer-verified method for quantifying fat in olestra-containing snack foods (JAOAC, 81, 848-868, 1998, “Determination of fat in olestra-30 containing savory snack products by capillary gas chromatography”, PVM 4:1995, AOAC International, Gaithersburg, Md.). The principle of this method involves extraction of the food product with chloroform-methanol solution, yielding a total lipid extract that contains the digestible fat and any non-digestible lipid. The lipid extract is hydrolyzed by lipase, yielding fatty acids from the digestible fat. The fatty acids are precipitated as calcium soaps and the isolated fatty acid soaps are converted back into fatty acids with hydrochloric acid and extracted into hexane. The isolated fatty acids are converted to methyl esters with boron trifluoride-methanol solution and quantified by capillary gas chromatography.

a.) The digestible fat and saturated fat content per a given mass of food is calculated as follows:

gdigestible fat=(mass of food in grams)×(%digestible fat/100)

gdigestible saturated fat=(mass of food in grams)×(% digestible saturated fat/100)

b.) Calories from digestible fat and saturated fat are calculated by multiplying by 9:

Energy from fat(kcal)=(gdigestible fat)×9 kcal/g

Energy form saturated fat(kcal)=(gdigestible saturated fat)×9 kcal/g

2. Extractable Lipid and Calculation of Non-Digestible Lipid: The total extractable lipid content of a food is measured by an extraction method known as AOAC Official Method 983.23, “Fat in Foods; Chloroform-Methanol Extraction Method” (45.4.02, Chp. 45, pg. 64-65). Percent total non-digestible lipid is calculated as follows:

(%non-digestible lipid)=(%extractable lipid)−(%digestible fat)

The percent digestible fat value in the above equation is derived from method #1 of Applicants' Analytical Protocols.

The non-digestible lipid content per a given mass of food is calculated as follows:

(gnon-digestible lipid)=(mass of food in grams)×(%non-digestible lipid/100)

3. Beta-Glucan Soluble Fiber: The content of beta-glucan soluble fiber in a food is measured by an enzymatic-spectrophotometric method according to AOAC Official Method 992.28, “(1→3)(1→4)-Beta-D-Glucans in Oat and Barley Fractions and Ready-to-Eat Cereals” (32.2.06, Chp. 32, pg. 28-29C).

The beta-glucan soluble fiber content per a given mass of food is calculated as follows:

(gbeta-glucan soluble fiber)=(mass of food in grams)×(%beta-glucan soluble fiber/100)

4. Water Activity: The water activity (Aw) of a food is measured using the following protocol and instruments:

Principle: The Rotronic Hygroskop relative humidity meter uses probes, each containing a humidity sensor and a temperature sensor, to measure the equilibrium relative humidity above a sample. A sample is introduced to the probe in an air tight chamber. After equilibrium has been reached, the relative humidity reading obtained from the instrument can be used to determine water activity (Aw).

Apparatus

a.) Rotronic Hygroskop model DT Relative Humidity Meter

b.) Model DMS100H Humidity Cells

c.) Rotronic Sample Dishes Part # PS-14

Reagents and Solutions

a.) 35% RH standard solution (EA-35) supplied by Rotronic Instrument Corp.

b.) 50% RH standard solution (EA-50) supplied by Rotronic Instrument Corp.

c.) 65% RH standard solution (EA-65) supplied by Rotronic Instrument Corp.

d.) 80% RH standard solution (EA-80) supplied by Rotronic Instrument Corp.

Procedure

a.) Instrument Operation and Calibration

(i) Prepare a standard curve of meter reading vs. % relative humidity (% RH) at 25° C. using the four RH standards listed in this method. The accuracy of the calibration curves should be checked periodically using the relative humidity standard solutions.

(ii) Carefully open a vial of RH standard solution and pour the contents into a plastic sample dish. Place the sample dish containing the standard solution into cell #1 of the instrument and seal tightly. Allow at least one hour for the meter reading to stabilize. Record the meter and temperature readings.

(iii) Repeat step 2 for the other humidity standards.

(iv) Prepare a standard curve by plotting the meter readings against the known RH of the standards.

(v) Prepare a standard curve for cell #2 in the same fashion.

b.) Sample Analysis

(i) Select a humidity cell to use for the analysis. Wipe clean the inner surfaces of the cell with a paper towel. This will remove anything left over from a previous sample.

(ii) Obtain a sample of food product. Samples must be at room temperature before the analysis can be run.

(iii) Place the sample into a plastic sample dish. The sample may need to be crushed or ground (eg. crackers) to fit into the dish. The dish should be filled as much as possible with the sample.

(iv) Place the sample dish into a cell and place the cell into the instrument. Keeping the cell level, seal the cell tightly to the instrument.

(v) Allow at least ½ hour for meter reading to stabilize. Trend lights on both the RH meter and temperature meter should not be lit when recording a reading. If either is lit at the end of ½ hour, wait until they go out before recording the meter readings.

(vi) Record the RH and temperature meter readings.

(vii)Convert the RH meter reading to the equilibrium % RH using the previously prepared standard curve for the cell used. Convert the equilibrium relative humidity to Aw.

c.) Water activity (Aw) Calculations: Aw=% RH/100

All AOAC (Association of Official Analytical Chemists) published methods can be found in the following reference which is incorporated by reference in its entirety:

AOAC International, Official Methods of Analysis, P. Cunniff (ed.), 16^{th} edition, 5^{th} Revision, 1999, Gaithersburg, Md.

### PRODUCT AND PROCESS EXAMPLES

The following examples and processing teachings are illustrative of the invention and are not to be construed to limit the invention in any way.

### Example 1

Beta-glucan soluble fiber and non-digestible fat mixture

| | |
|---|---|
| Ingredient | Weight percent |

| | |
|---|---|
| Olestra (Olean ® brand, The Procter & Gamble | 66.7 |
| Co., Cincinnati, OH) | |
| * High beta-glucan Oatrim ® (Rhone Poulenc | 33.3 |
| Food Ingredients, Cranbury, NJ) | |

* High beta-glucan Oatrim ® fiber analysis: beta-glucan soluble fiber = 18.5% minimum (dry basis)

Making Procedure

The olestra and high beta-glucan Oatrim® are blended together to form a semi-solid composition. First, the olestra is melted by heating to a completely molten state at approximately 150° F. (65.6° C.). High beta-glucan Oatrim® is then combined with the molten olestra and uniformly dispersed by mixing. The olestra/Oatrim blend is then poured into 4 fl. oz. (118.3 ml) glass jars and allowed to cool to room temperature (70° F., 21.1° C.) to form a semi-solid composition. The resulting semi-solid composition comprises approximately 20 g non-digestible fat (olestra) and approximately 1.85 g beta-glucan soluble fiber per 30 g serving.

Where necessary for a patient's health or required by regulation, the finished composition is fortified with vitamins. In the United States, the beta-glucan soluble fiber and non-digestible fat mixture is fortified with a minimum of 170 IU of vitamin A per gram of Olean®; 12 IU of vitamin D per gram of Olean® and 8 μg of vitamin K per gram of Olean®. Said fortification is accomplished by combining a vitamin source such as Vitamin A, D₃, K₁ blend, that is supplied by Watson Foods Co., West Haven, Conn., with said beta-glucan soluble fiber and non-digestible fat mixture.

Method of Use

The composition is used as an ingredient in the preparation of baked goods, such as cookies, cakes, crackers, and muffins; or it is ingested as a 30 g unit dosage to lower serum cholesterol. Two such unit doses are orally ingested each day, preferably with meals on a chronic basis, to reduce total and/or LDL cholesterol at least 5%.

### Example 2

Cheddar cheese filled crackers having a
crumb to filling ratio by weight of 1.5:1

| | Crumb | Filling |
|---|---|---|
| | Formula | Formula |
| | weight | weight |
| Ingredient | percent | percent |
| 62DE Corn Syrup (Quality Ingredients Corp., | 0.62 | |
| Chester, N. J.) | | |
| Olean ® (Procter & Gamble Co., Cincinnati, OH.) | 9.13 | 32.00 |
| Malt Syrup (Hawkeye 5900, Quality Ingredients | 1.24 | |
| Corp., Chester N. J.) | | |
| Granulated Sugar (Holly Sugar Co., Worland, | 5.60 | |
| WY.) | | |
| Salt - TFC Purex (Morton International, Inc., | 0.30 | |
| Philadelphia, PA.) | | |
| L-Cysteine HCl Monohydrate (Quality Ingredients | 0.04 | |
| Corp., Chester N. J.) | | |
| Vitamin A, D₃, K₁ blend (Watson Foods Co., West | 0.06 | 0.07 |
| Haven, CT.) | | |
| Flour - soft wheat (Siemer Milling Co., | 33.99 | |
| Teutopolis, IL.) | | |
| Fiber - soluble (Fibersol-2, Matsutani Chem. Ind., | | 8.00 |
| Itami-city Hyogo, Japan) | | |
| Isolated Soy Protein (Supro ® 661, Protein | 4.40 | 2.50 |
| Technologies Intl., St. Louis, MO.) | | |
| Sodium Bicarbonate (Church & Dwight Co., | 0.95 | |
| Princeton, NJ.) | | |
| Calcium Phosphate Monobasic (Regent 12XX, | 0.76 | |
| Rhodia, Cranbury, N. J.) | | |
| Sodium Aluminum Phosphate (Levair, Rhodia, | 0.76 | |
| Cranbury, N. J.) | | |
| Ammonium Bicarbonate (Church & Dwight Co., | 2.40 | |
| Princeton, NJ.) | | |
| Whey Protein Isolate (BiPRO, Davisco Food | | 10.00 |
| International, Inc., Le Sueur, MN.) | | |
| Oat Bran Conc. (Oatcor, Quaker Oats Co., | 16.89 | 11.50 |
| Chicago, IL.) | | |
| Water | 22.86 | |
| Corn Syrup Solids (M200, Grain Processing Corp., | | 10.00 |
| Muscatine, IA.) | | |
| Cheese Powder (#2100078346, Kraft Foods | | 23.93 |
| Ingredients, Memphis, TN.) | | |
| Cheese Flavor (#1030WYF, Edlong Corporation, | | 2.00 |
| Elk Grove Village, IL.) | | |

Cheese Filling Making Procedure

1. The fiber and the primary source of beta glucan soluble fiber (oat bran concentrate) are weighed in a separate bowl.

2. Any cheese powder, soy protein, whey protein, corn syrup solids, sucrose, and cheese flavor are weighed together.

3. Next, the Olean® and Kaomel Flakes are weighed and then mixed together in a container.

4. The Olean® and Kaomel Flake mixture is melted by heating until the temperature reaches 150° F. 160° F. (65.6° C.-71.1° C.). For lab scale, this is best accomplished by heating in a microwave oven at one-minute intervals, with stirring in between intervals, with power setting on high. After the desired temperature is reached, the vitamins are added.

5. The melted fat blend is mixed with the materials from Step #1 using a Kitchen Aid (Model KSM90 Ultra Power) mixer for 1 minute at speed setting #2. The rest of the dry ingredients are added and blended for 5 minutes at speed setting #5. 6. Then the mixture is cooled through the temperature range of 130° F.-140° F. (54.4° C.-60.0° C.) in about 10 minutes to ensure the proper crystallizing structure. This can usually be accomplished by ambient cooling for lab batch sizes.

7. The resulting filling is stored until used.

Cracker Making Procedure

Dough Making

1. Corn syrup, malt syrup, shortening, hot water at 160° F. (71.1° C.), and enzyme tablets dissolved in water are weighed into an APV 100# single blade horizontal mixer and then mixed for 30 seconds at 38 rpm.

2. Next, sugar, salt, vitamin blend, and L-cysteine are weighed into the mixer and then mixed for 2 minutes at 38 rpm.

3. Then the remaining dry ingredients (flour, fibers, proteins, sodium bicarbonate, and non-ammonia leavening salts) are weighed into mixer and mixed for 3 minutes at 45 rpm.

4. Then ammonium bicarbonate, dissolved in cool water, is added and mixed for one minute at 60 rpm.

5. The resulting dough is emptied into a stainless steel tram, covered with plastic sheet, and allowed to “rest” at room temperature for 30 minutes.

Dough Forming

1. Dough is fed through a three-roll mill having two initial 16.5 inch (41.9 cm) corrugated rolls and one smooth 11.8-inch (30.0 cm) diameter roll and sheeted to 0.25 inches (0.64 cm). The take-off belt speed exiting the three-roll mill is 2.0 fpm (0.6 mpm), and is matched to the speed of the dough sheet as it exits the three-roll mill.

2. The sheet is sent through a calender roll #1 (an 11.8 inch or 30.0 cm diameter two-roll mill), and sheeted to approximately 0.10 inches (0.25 centimeters) in thickness. The take-off belt speed exiting the calender roll #1 is 4.4 fpm (1.34 mpm), and is matched to the speed of the dough sheet as it exits the calender roll #1.

3. As the sheet comes through calender roll # 1, it is folded over eight times to a width of approximately 10 inches (25.4 cm) to form a bundle of laminated dough. The bundle is covered with plastic film to prevent dehydration and briefly set aside while additional bundles are collected.

4. The laminated sheet of Step 3 above is sent through the two-roll mill #1 again to form a 0.10-inch (0.25 cm) thick sheet.

5. Before the dough sheet reaches calender roll #2 (an 11.8 inch or 30.0 cm diameter two-roll mill), bits, such as, but not limited to, pieces of nuts vegetables, grains, meats and candies, may optionally be added. These bits are uniformly sprinkled on the dough sheet immediately before calender roll #2 such that they are pressed into the dough sheet

6. The sheet continues on calender roll #2 to form a finished dough sheet approximately 0.08 inches (0.20 cm) thick. The take-off belt speed exiting the calender roll #2 is 7.9 fpm (2.41 mpm), and is matched to the speed of the dough sheet as it exits the calender roll #2.

7. The dough sheet is then passed under an embossing roller and under a cutter die roll to form crackers of desired size/shape. The belt speed is 7.7 fpm (2.35 mpm). The embossing roller is a 3.75-inch (9.52 cm) diameter roll with a uniform pattern of 0.061-inch (0.153 cm) diameter pins spaced 5/16 inches (0.794 centimeters) apart in both the axial and radial directions. The 3.875-inch (9.842 cm) diameter cutter roll (obtained from Weidenmiller Co. of Itasca, Ill.) can be designed to cut a variety of shapes. The shape used in these examples is a 1.4 inch (3.6) diameter round shape with docking holes. These docking pins serve the purpose of preventing the dough form from inflating during baking. The function of the docking pins is thought to join the dough layers together and create venting during baking.

8. After separating the web (the portion of the sheet left over after the shapes are cut out), the crackers are salted using a roller-salter or equivalent. The web may be recycled back to the dough waiting to be introduced into the three-roll mill.

9. The cracker dough forms are then sprayed with a water mist (flow rate=65-212 g/min.) before baking. This helps attain a lighter color after baking.

Cracker Baking

1. The cracker dough forms are transferred as a continuous feed from the dough-forming belt onto the oven band such that their relative spacing is undisturbed (a slight speed differential is permissible if it is desired to place the cracker dough forms closer or further apart on the oven band prior to baking). The oven band is made of metal of the open weave versus solid surface type. Solid surface metal oven bands may also be used for certain applications.

2. The cracker dough forms are baked in an APV 45 foot (13.7 m) long three-zone indirect-fired oven. Each zone has independent top and bottom heat applied. Dampers and temperatures in each zone are set at the following conditions:

| | | | |
|---|---|---|---|
| 1^{st} zone top: | 465° F. (240.6° C.), | bottom: | 500° F. (260.0° C.), |
| | | | damper closed |
| 2^{nd} zone top: | 480° F. (248.9° C.), | bottom: | 520° F. (271.1° C.), |
| | | | damper 1/2 |
| 3^{rd} zone top: | 355° F. (179.4° C.), | bottom: | 425° F. (218.3° C.), |
| | | | damper open |

| | |
|---|---|
| Oven band speed (fpm): | 11.0 (3.35 mpm) |

Final moisture contents are about 0-4%.

Post Baking

1. As the hot baked crackers exit the oven, they are sprayed with hot oil or Olean® at approximately 160° F. (71.1° C.) to a level of about 10% their post baked weight. The crackers are passed under heat lamps for approximately 15 seconds to aid in absorption of oil.

2. The crackers are then passed through a cooling tunnel at room temperature. Olean® containing products must cool through the temperature range of 130° F.-140° F. (54.4° C.-60.0° C.) in about 10 minutes to ensure the proper crystalline structure.

Sandwiching Procedure For Crackers

1. The filling is spread on a cracker.

2. A second cracker is placed on top of the filling that is spread on the first cracker thereby forming a finished sandwich cracker.

Results

The finished filled cracker product is analyzed according to the protocols disclosed in the “Analytical Protocols” section of this application and is found to contain approximately 7.5 g olestra per 30 g serving and approximately 0.6 g beta-glucan soluble fiber per 30 g serving.

Method of Use

The filled cracker of Example 2 is used as a functional food composition to lower serum total and LDL-cholesterol and to control postprandial blood glucose and insulin levels. The product contains approximately 7.5 g of olestra (Olean® brand, The Procter & Gamble Co., Cincinnati, Ohio) and about 0.6 g of oat beta-glucan soluble fiber per 30 g serving size. A group of at least 25 hypercholesterolemic subjects consume 4 servings/day of the filled crackers, thereby ingesting approximately 30 g olestra and 2.4 g beta-glucan soluble fiber per day. The servings are spaced throughout the day; e.g., consumed with the breakfast, lunch, and dinner meals, and as a between meal snack. Consumption continues for a period of 28 consecutive days. On day 1, a fasting blood sample is collected from each subject for measurement of the baseline blood lipid profile (total, LDL-, and HDL-cholesterol, and total lipids). On day 28, a second fasting blood sample is drawn from each subject and the blood lipid profile measured. For each subject, the blood lipid profile on day 28 is compared to the baseline profile measured on day 1. Following treatment, the total and/or LDL-cholesterol is reduced from the baseline level by an average of at least 5%.

### Example 3

Direct extruded cheese filled snack product having
a crumb to filling ratio by weight of 1.5:1

| | Crumb | Filling |
|---|---|---|
| | Formula | Formula * |
| | weight | weight |
| Ingredient | percent | percent |
| Olean ® (Procter & Gamble Co., Cincinnati, OH.) | | 33.00 |
| Oat Bran Conc. (Oatcor ®, Quaker Oats Co., | 22.33 | 15.00 |
| Chicago, IL.) | | |
| Sugar 12X (Amalgamated Sugar Co., Ogden, UT.) | 2.00 | |
| Salt - Flour Salt (Cargil Inc., St. Clair, MI.) | 1.40 | |
| Instant Clearjel Starch (National Starch & | 18.00 | |
| Chemical, Bridgewater, NJ.) | | |
| Maltrin M100 Maltodextrin (Grain Processing | 4.00 | |
| Corp., Muscatine, IA.) | | |
| Starch, Baka Plus (National Starch & Chemical, | 5.00 | |
| Bridgewater, NJ.) | | |
| Onion Powder (Basic Vegetable Products, Inc., | 0.74 | |
| Suisun, CA.) | | |
| Fiber - soluble (Fibersol-2, Matsutani Chem. Ind., | | 5.00 |
| Itami-city Hyogo, Japan) | | |
| Isolated Soy Protein (Supro ® 661, Protein | | 5.50 |
| Technologies Intl., St. Louis, MO.) | | |
| Sodium Bicarbonate (Church & Dwight Co., | 0.55 | |
| Princeton, NJ.) | | |
| Whey Protein Isolate (BiPRO, Davisco Food | | 10.00 |
| International, Inc., Le Sueur, MN.) | | |
| Yellow Masa (Lauhoff Grain Co., Danville, IL.) | 45.98 | |
| Cheese Powder (#2100078346, Kraft Foods | | 25.00 |
| Ingredients, Memphis, TN.) | | |
| Corn Syrup Solids (M200, Grain Processing Corp., | | 6.50 |
| Muscatine, IA.) | | |
| * Where necessary for a patient's health or required by regulation, the filled extruded snack product is fortified with vitamins. In the United States, the beta-glucan soluble fiber and non-digestible fat mixture is fortified with a minimum of 170 IU of vitamin A per gram of Olean ®; 12 IU of vitamin D per gram of Olean ® and 8 μg of vitamin K per gram of Olean ®. Said fortification is accomplished by combining a | | |
| #vitamin source such as Vitamin A, D₃, K₁ blend, that is supplied by Watson Foods Co., West Haven, CT., with said filling materials as described in the Cheese Filling Making Procedure below. | | |

### Making Procedures

Dough Making:

1. Each ingredient is weighed and then combined in a 150 lb (68.2 kg) horizontal ribbon blender.

2. Next, the mixture of ingredients is blended for 15 minutes to form a dry dough mix and then transferred into a food grade container for temporary storage.

Extrusion Process:

1. The dry dough mix is added to the feeder bin (hopper) of a K-Tron loss in weight feeder, which is calibrated to 378 g/min (±5 g). The feeder transfers the dry mix to the pre-mixer of a Pavan single screw extruder (Model F70 Extruder Former).

2. In the pre-mixer, water is added at a rate of 0.37 lbs/min. (0.17 kg/min.) while at ambient temperature.

3. The emulsifier, Panodan SDK (Danisco, Copenhagen, Denmark), is then added to the pre-mixer at a rate and temperature of 5 g/min. and 150° F. (65.6° C.).

4. The dough is then mechanically fed by the pre-mixer into the main mixer where it is further mixed, cooled and moved toward the extrusion screw.

5. At this point the single screw extruder pulls the dough into the screw chamber where the dough is forced though a die housing to give it shape. The dough is then cut via rotating blades to produce individually sized pieces.

Frying:

1. The extruded product (extrudate) of Step #5 above is placed in a frying basket that is then placed into a 50 lb (22.7 kg) fryer containing 100% Olean® at 350° F. (176.7° C.). The extrudate is free fried (i.e., on the oil surface) for 30 seconds and then submersed and fried for an additional 60 seconds.

2. The extrudate is then transferred from the fryer to a paper towel where it is allowed to cool. The extruded product has approximately a 20% Olean® content after frying.

Cheese Filling Making Procedure

1. The fiber and the primary source of beta glucan soluble fiber (oat bran concentrate) are weighed in a separate bowl.

2. Any cheese powder, soy protein, whey protein, corn syrup solids, sucrose, and cheese flavor are weighed together.

3. Next, the Olean® and Kaomel Flakes are weighed and then mixed together in a container.

4. The Olean® and Kaomel Flake mixture is melted by heating until the temperature reaches 150° F. 160° F. (65.6° C.-71.1° C.). For lab scale, this is best accomplished by heating in a microwave oven at one-minute intervals, with stirring in between intervals, with power setting on high. After the desired temperature is reached, any vitamins are added.

5. The melted fat blend is mixed with the materials from Step #1 using a Kitchen Aid (Model KSM90 Ultra Power) mixer for 1 minute at speed setting #2. The rest of the dry ingredients are added and blended for 5 minutes at speed setting #5.

6. Then the mixture is cooled through the temperature range of 130° F.-140° F. (54.4° C.-60.0° C.) in about 10 minutes to ensure the proper crystallizing structure. This can usually be accomplished by ambient cooling for lab batch sizes.

7. The resulting filling is stored until used.

Filling Procedure:

1. After frying, random snack pieces are weighed to obtain an average weight, which is about 1.1 g per snack piece.

2. A snack to filling ratio of about 1.5:1 is used.

3. The filling is added to the snack pieces using a spatula to force the filling into the void spaces in the snack.

4. The filled snack pieces are seasoned with Nacho seasoning (Kerry Ingredients, Beloit Wis.) by placing about 100 g of snack pieces and 3 g of seasoning in a plastic container with a lid and shaking the snack pieces in the container until the pieces are fully covered.

Results

The finished filled snack product is analyzed according to the protocols disclosed the “Analytical Protocols” section of this application and is found to contain approximately 7.6 g olestra per 30 g serving and approximately 0.6 g beta-glucan soluble fiber per 30 g serving.

### Example 4

Potato Crisp

| | |
|---|---|
| | Formula |
| Ingredient | weight percent |

| | |
|---|---|
| Potato Flakes | 35.97 |
| (Basic American Foods, Blackfoot, ID) | |
| Potato Granules | 5.34 |
| (Basic American Foods, Blackfoot, ID) | |
| Modified Corn Starch, N-Creamer | 0.59 |
| (National Starch and Chemical Company, | |
| Bridgewater, NJ) | |
| Concentrated Oat Bran, * Oatcor ® Brand | 22.00 |
| (Quaker Oats Company, Chicago, IL) | |
| High Amylose Starch, Hylon VII (˜70% amylose) | 3.27 |
| (National Starch and Chemical Company, | |
| Bridgewater, NJ) | |
| Potato Peel (skin) | 0.98 |
| (Basic American Foods, Blackfoot, ID) | |
| Emulsifier | 0.60 |
| (Blend of olestra, monoglyceride, and | |
| polyglycerol ester**) | |
| Ascorbic Acid | 0.03 |
| (Roche Vitamins Inc., Parsippany, NJ) | |
| Potato flavor (Firmenich Inc., Plainsboro, NJ) | 0.04 |
| Water | 31.18 |
| | 100.0 |

* Concentrated oat bran fiber analysis: total dietary fiber = 20% minimum (dry basis), beta-glucan soluble fiber = 11.5% minimum (dry basis)
**Emulsifier is a blend of 85% olestra (Olean brand, The Procter & Gamble Co., Cincinnati, OH), 12.75% Dimodan O distilled monoglyceride (Danisco Ingredients, Inc., New Century, KS), and 2.25% DHBM polyglycerol ester (Lonza, Williamsport, PA).

Dough Making:

1. The potato flakes, potato granules, modified corn starch, concentrated oat bran, high amylose starch and potato peel are weighed, combined and put into a food processor (Waring commercial food processor) and mixed for 1 minute.

2. Water is heated to approximately 180° F. (82.2° C.) and combined with the emulsifier, potato flavor, and ascorbic acid using a high shear mixer for 15 seconds. During this mixing process the temperature of the blend is dropped, therefore, the temperature is adjusted to 160° F.±5° F. (71.1° C.±2.9° C.) by heating using a microwave oven.

3. While the food processor is on, the liquid mixture of Step #2 above is combined with the dry ingredients of Step # 1 above and the resulting mixture is mixed for 30 seconds.

4. Next the processor is stopped and its sides are scraped with a spatula to loosen any adhered material.

The processor is then restarted and the mixture is mixed for another 30 seconds to form a dough.

5. The dough of Step # 4 above is then transferred into a sealable plastic bag to minimize moisture loss.

6. Next, the dough is transferred to a roll mill and roll milled to a thickness of 0.023-0.026 inches (0.58-0.66 mm).

7. Then, 1.70″ (4.32 cm) diameter circles are cut from the dough sheets, using a circular cookie cutter.

Frying:

1. The dough circles are fried for 9 seconds, using a stainless steel carrier that holds 6 circular dough pieces, in a 50 lb (22.7 kg) oil capacity foodservice fryer (Frymaster Corp., Shreveport, La.) filled with an 85/15 blend of olestra (Olean®, The Procter & Gamble Co., Cincinnati, Ohio) and cottonseed triglyceride oil maintained at 375° F. (190.6° C.).

2. During frying the dough circles become crisps, and after frying the crisps are removed from the carrier and allow them to cool on a paper towel.

Salting and Vitamin Fortification:

1. The crisps are weighed and put into a seasoning drum.

2. The crisps are salted to a level of 1.3% in the seasoning drum and then packaged in sealed, foil laminated bags.

Where necessary for a patient's health or required by regulation, the finished crisps are fortified with vitamins. In the United States, the finished crisps are fortified with a minimum of 170 IU of vitamin A per gram of Olean®; 12 IU of vitamin D per gram of Olean® and 81 g of vitamin K per gram of Olean®. Said fortification is accomplished by combining a vitamin source such as Vitamin A, D₃, K, blend, that is supplied by Watson Foods Co., West Haven, Conn., with salt in the seasoning drum, wherein the desired level of salt and vitamins are applied to the surface of the crisps.

Results

The resulting finished potato crisps are analyzed according to the protocols disclosed the “Analytical Protocols” section of this application and are found to contain approximately 7.5 g olestra per 30 g serving and approximately 0.8 g beta-glucan soluble fiber per 30 g serving.

Method of Use

The potato crisps are a very palatable dietary option for lowering blood cholesterol and controlling blood glucose and insulin levels. Four 30 g servings of the crisps are consumed on a daily basis for at least 28 consecutive days, thereby delivering a daily intake of about 30 g olestra and about 3.2 g beta-glucan soluble fiber.

### Example 5

Potato Crisps

| | Formula |
|---|---|
| Ingredient | weight percent |
| Potato Flakes | 35.97 |
| (Basic American Foods, Blackfoot, ID) | |
| Potato Granules | 5.34 |
| (Basic American Foods, Blackfoot, ID) | |
| Modified Corn Starch, N-Creamer | 0.59 |
| (National Starch and Chemical Company, | |
| Bridgewater, NJ) | |
| * High Beta-Glucan Oatrim | 22.00 |
| (Rhone Poulenc Food Ingredients, Cranbury, NJ) | |
| High Amylose Starch, Hylon VII (˜70% amylose) | 3.27 |
| (National Starch and Chemical Company, | |
| Bridgewater, NJ) | |
| Potato Peel (skin) | 0.98 |
| (Basic American Foods, Blackfoot, ID) | |
| Emulsifier | 0.60 |
| (blend of olestra, monoglyceride, and polyglycerol | |
| ester**) | |
| Ascorbic Acid | 0.03 |
| (Roche Vitamins, Inc., Parsippany, NJ) | |
| Potato flavor (Firmenich, Inc., Plainsboro, NJ) | 0.04 |
| Water | 31.18 |

* High beta glucan Oatrim fiber analysis: beta glucan soluble fiber = 18.5% minimum (dry basis)
**Emulsifier is a blend of 85% olestra (Olean brand, The Procter & Gamble Co., Cincinnati, OH), 12.75% Dimodan O distilled monoglyceride (Danisco Ingredients, Inc., New Century, KS), and 2.25% DHBM polyglycerol ester (Lonza, Williamsport, PA).

Dough Making:

1. The potato flakes, potato granules, modified corn starch, high beta-glucan Oatrim, high amylose starch, and potato peel are weighed, combined and put into a food processor (Waring commercial food processor) and mixed for 1 minute.

2. Water is heated to approximately 180° F. (82.2° C.) and combined with the emulsifier and ascorbic acid, using a high shear mixer for 15 seconds. During this mixing process the temperature of the blend is dropped, therefore, the temperature is adjusted to 160° F.±5° F. (71.1° C.±2.9° C.) by heating using a microwave oven.

3. While the food processor is on, the liquid mixture of Step #2 above is combined with the dry ingredients of Step # 1 above and the resulting mixture is mixed for 30 seconds.

4. Next the processor is stopped and its sides are scraped with a spatula to loosen any adhered material.

The processor is then restarted and the mixture is mixed for another 30 seconds to form a dough.

5. The dough of Step # 4 above is then transferred into a sealable plastic bag to minimize moisture loss.

6. Next, the dough is transferred to a roll mill and roll milled to a thickness of 0.023-0.026 inches (0.58-0.66 mm).

7. Then, 1.70″ (4.32 cm) diameter circles are cut from the dough sheets, using a circular cookie cutter.

Frying:

1. The dough circles are fried for 9 seconds, using a stainless steel carrier that holds 6 circular dough pieces, in a 50 lb (22.7 kg) oil capacity foodservice fryer (Frymaster Corp., Shreveport, La.) filled with olestra (Olean®, The Procter & Gamble Co., Cincinnati, Ohio) maintained at 375° F. (190.6° C.).

2. During frying the dough circles become crisps, and after frying the crisps are removed from the carrier and allow them to cool on a paper towel.

Salting and Vitamin Fortification:

1. The crisps are weighed and put into a seasoning drum.

2. The crisps are salted to a level of 1.3% in the seasoning drum and then packaged in sealed, foil laminated bags.

Where necessary for a patient's health or required by regulation, the finished crisps are fortified with vitamins. In the United States, the finished crisps are fortified with a minimum of 170 IU of vitamin A per gram of Olean®; 12 IU of vitamin D per gram of Olean® and 8 μg of vitamin K per gram of Olean®. Said fortification is accomplished by combining a vitamin source such as Vitamin A, D₃, K, blend, that is supplied by Watson Foods Co., West Haven, Conn., with salt in the seasoning drum, wherein the desired level of salt and vitamins are applied to the surface of the crisps.

Results:

The finished potato crisps are analyzed according to the protocols disclosed the “Analytical Protocols” section of this application and are found to contain approximately 9 g olestra per 30 g serving, and 1.2 g beta-glucan soluble fiber per 30 g serving.

Method of Use

The potato crisps are used as a palatable dietary treatment to lower serum total and/or LDL cholesterol, and to control postprandial blood glucose and insulin levels. Four 30 g servings per day are ingested, preferably on a chronic basis, thereby delivering approximately 36 g olestra and 4.8 g beta-glucan soluble fiber per day. The servings are spaced throughout the day; i.e., consumed with the breakfast, lunch, and dinner meals and as a between meal snack. After 28 days of consumption, the serum total and/or LDL cholesterol level is reduced by at least 5%.

### Example 6

Cracker

| | Formula |
|---|---|
| Ingredient | weight percent |
| Corn syrup, 62DE (Quality Ingredients Corp., | 0.62 |
| Chester, NJ) | |
| Malt syrup, Hawkeye 5900 (Quality Ingredients | 1.24 |
| Corp., Chester, NJ) | |
| Olestra (Olean ® brand; Procter & Gamble Co., | 9.13 |
| Cincinnati, OH) | |
| Trem-Tabs, proteolytic enzyme tablet | 0.01 |
| (Cain Food Industries, Inc., Dallas, TX) | |
| Sugar, granulated (Holly Sugar Co., Worland, | 5.6 |
| WY) | |
| Salt, TFC Purex (Morton International, Inc., | 0.3 |
| Philadelphia, PA) | |
| L-Cysteine hydrochloride, monohydrate (Quality | 0.04 |
| Ingredients Corp., Chester, NJ) | |
| Vitamin A, D₃, K₁ combination powder (Watson | 0.06 |
| Foods Co., West Haven, CT) | |
| Flour, soft wheat, 5-9% protein (Siemer Milling | 34.02 |
| Co., Teutopolis, IL) | |
| Oat Bran Concentrate, Oatcor ® brand* | 21.28 |
| (The Quaker Oats Company, Chicago, IL) | |
| Sodium bicarbonate (Church & Dwight Co., | 0.95 |
| Princeton, NJ) | |
| Calcium phosphate monobasic, Regent 12XX | 0.76 |
| (Rhodia, Cranbury, NJ) | |
| Sodium aluminum phosphate, Levair (Rhodia, | 0.76 |
| Cranbury, NJ) | |
| Ammonium bicarbonate (Church & Dwight Co., | 2.4 |
| Princeton, NJ) | |
| Water | 22.83 |

*Concentrated oat bran fiber analysis: total dietary fiber = 20% minimum (dry basis); beta-glucan soluble fiber = 11.5% minimum (dry basis)

Dough Making:

1. The corn syrup, malt syrup, olestra, enzyme tablet, and 87.3% of the water (hot) are combined in a APV 100 lb. (45 kg) horizontal blade mechanical mixer and mixed for 30 seconds at 38 RPM.

2. Next, the sugar, salt, L-cysteine hydrochloride and vitamin ADK powder are added to the mixer and mixed for 120 seconds at 38 RPM.

3. Then the flour, oat bran concentrate, sodium bicarbonate, calcium phosphate monobasic, and sodium aluminum phosphate are added to the mixer and mixed for 180 seconds at 45 RPM.

4. Next, the ammonium bicarbonate is dissolved in the remaining water (at room temperature) and this solution is added to the mixer and the resulting dough is mixed for 60 seconds at 60 RPM.

5. The dough is then allowed to rest for 30 minutes at room temperature.

Lamination:

1. The dough of Step # 5 above is fed into a 3 roll mill by hand. The dough sheet exits the 3 roll mill having a thickness of approximately 0.18 inches (4.57 mm). The resulting dough sheet is then fed through a 2 roll gauge mill and exits with a thickness of approximately 0.08 inches (2.03 mm).

2. The dough of Step # 1 is then folded back on itself in about 9 inch (22.86 cm) lengths. After a total of 8 folds are made, the laminated dough is cut away from the dough exiting the roll mill.

Cracker Making:

1. A laminated dough section from Step # 2 above is re-fed into the 2 roll gauge mill by hand and is then passed through a 2 roll sheeter. After exiting the sheeter, the dough is approximately 0.07 inches (1.78 mm) thick.

2. Next, the sheeted dough is moved under an embossing roll and to the cutter/docker where the individual cracker shapes are cut from the dough.

3. Then the unused dough webbing is removed and the cut dough pieces are passed under a salter and a water mist sprayer.

4. Next, the cut dough pieces enter a three zone oven to be baked.

5. After baking, the crackers are sprayed with a hot (approximately 160-180° F. or 71-82° C.) olestra/triglyceride oil blend (50:50) at a level of approximately 11.5% and proceed through a cooling tunnel where they are ambiently cooled.

6. Upon exiting the cooling tunnel, the crackers are collected and packaged.

Settings

| | |
|---|---|
| Cracker size (diameter) | 1.4 inches |
| Salter belt speed | 8 fpm (2.4 mpm) |
| Salter output | 14.4 g/min |
| Salt level | 18.5 mg/cracker |
| Oven belt speed | 11.8 fpm (3.6 mpm) |
| Oven - Zone 1 - top | 500° F. (260° C.) |
| Oven - Zone 1 - bottom | 520° F. (271° C.) |
| Damper - Zone 1 | Closed |
| Oven - Zone 2 - top | 480° F. (249° C.) |
| Oven - Zone 2 - bottom | 500° F. (260° C.) |
| Damper - Zone 2 | ¾ Open |
| Oven - Zone 3 - top | 355° F. (179° C.) |
| Oven - Zone 3 - bottom | 425° F. (218° C.) |
| Damper - Zone 3 | Open |

Results

The finished cracker product is analyzed according to the protocols disclosed the “Analytical Protocols” section of this application and is found to contain approximately 5.1 g olestra per 30 g serving and approximately 0.9 g beta-glucan soluble fiber per 30 g serving

Method of Use

The crackers are used as a palatable dietary treatment to lower serum total and/or LDL cholesterol, and to control postprandial blood glucose and insulin levels. Four 30 g servings per day are ingested, preferably on a chronic basis, thereby delivering approximately 20.4 g olestra and 3.6 g beta-glucan soluble fiber per day. The servings are spaced throughout the day; i.e., consumed with the breakfast, lunch, and dinner meals and as a between meal snack.

### Example 7

Extruded snack product

| | Formula |
|---|---|
| Ingredient | weight percent |
| Yellow masa, Regular #0 (Azteca Milling, Irving, | 45.98 |
| TX) | |
| Starch, Instant Clearjel (National Starch & | 18.00 |
| Chemical Co., Bridgewater, NJ) | |
| Maltodextrin, Maltrin 100 (Grain Processing | 4.00 |
| Corp., Muscatine, IA) | |
| Fine granular sugar (Amalgamated Sugar Co., | 2.00 |
| Ogden, UT) | |
| Salt-flour salt (Cargill Foods, Inc., St. Clair, MI) | 1.40 |
| Onion powder (Basic Vegetable Products, Suisun, | 0.74 |
| CA) | |
| Sodium bicarbonate (Church & Dwight Co., | 0.55 |
| Princeton, NJ) | |
| Starch, Baka Plus (National Starch & Chemical | 5.00 |
| Co., Bridgewater, NJ) | |
| Oat Bran Concentrate, Oatcor ® brand* | 22.33 |
| (The Quaker Oats Company, Chicago, IL) | |
| | 100.0 |

*Concentrated oat bran fiber analysis: total dietary fiber = 20% minimum (dry basis); beta-glucan soluble fiber = 11.5% minimum (dry basis)

### Making Procedures

Dough Making:

1. Each ingredient is weighed and then combined in a 150 lb (68.2 kg) horizontal ribbon blender.

2. Next, the mixture of ingredients is blended for 15 minutes to form a dry dough mix and then transferred into a food grade container for temporary storage.

Extrusion Process:

1. The dry dough mix is added to the feeder bin (hopper) of a K-Tron loss in weight feeder, which is calibrated to 378 g/min (±5 g). The feeder transfers the dry mix to the pre-mixer of a Pavan single screw extruder (Model F70 Extruder Former).

2. In the pre-mixer, water is added at a rate of 0.37 lbs/min. (0.17 kg/min.) while at ambient temperature.

3. The emulsifier, Panodan SDK (Danisco, Copenhagen, Denmark), is then added to the pre-mixer at a rate and temperature of 5 g/min. and 150° F. (65.6° C.).

4. The dough is then mechanically fed by the pre-mixer into the main mixer where it is further mixed, cooled and moved toward the extrusion screw.

5. At this point the single screw extruder pulls the dough into the screw chamber where the dough is forced though a die housing to give it shape. The dough is then cut via rotating blades to produce individually sized pieces.

Frying:

1. The extruded product (extrudate) of Step #5 above is placed in a frying basket that is then placed into a 50 lb (22.7 kg) fryer containing 100% Olean® at 350° F. (176.7° C.). The extrudate is free fried (i.e. on the oil surface) for 30 seconds and then submersed and fried for an additional 60 seconds.

2. The extrudate is then transferred from the fryer to a paper towel where it is allowed to cool. The extruded product has approximately a 20% Olean® content after frying.

Salting and Vitamin Fortification:

1. The product is weighed and put into a seasoning drum.

2. The product is salted to a level of 1.3% in the seasoning drum and then packaged in sealed, foil laminated bags.

Where necessary for a patient's health or required by regulation, the finished product is fortified with vitamins. In the United States, the finished product is fortified with a minimum of 170 IU of vitamin A per gram of Olean®; 12 IU of vitamin D per gram of Olean® and 8 μg of vitamin K per gram of Olean®. Said fortification is accomplished by combining a vitamin source such as Vitamin A, D₃, K, blend, that is supplied by Watson Foods Co., West Haven, Conn., with salt in the seasoning drum, wherein the desired level of salt and vitamins are applied to the surface of the product.

Results

The finished extruded snack product is analyzed according to the protocols disclosed the “Analytical Protocols” section of this application and is found to contain approximately 6.0 g olestra per 30 g serving and approximately 0.6 g beta-glucan soluble fiber per 30 g serving.

What is claimed:

1.) An orally-administered composition of matter for reducing blood cholesterol levels and controlling postprandial blood glucose and insulin levels in humans or lower animals, comprising a mixture of:
(a) beta-glucan soluble fiber or a source of beta-glucan soluble fiber; and
(b) a non-digestible fat or a source of non-digestible fat.

2.) The composition of claim 1 having a weight ratio of beta-glucan soluble fiber to non-digestible fat of from about 1:20 to about 20:1.

3.) The composition of claim 2 wherein the weight ratio of beta-glucan soluble fiber to non-digestible fat is from about 1:10 to about 10:1.

4.) The composition of claim 3 wherein the weight ratio of beta-glucan soluble fiber to non-digestible fat is from about 1:3 to about 3:1.

5.) The composition of claim 1 wherein said composition is a food comprising, on a single reference serving basis:
a.) at least about 0.5 grams of beta-glucan soluble fiber; and
b.) at least about 1 gram of non-digestible fat.

6.) The food of claim 5 wherein said food comprises at least about 0.75 grams of beta-glucan soluble fiber.

7.) The food of claim 5 wherein said food comprises:
a.) from about 0.5 grams to about 7.5 grams of beta-glucan soluble fiber; and
b.) from about 1 gram to about 16.0 grams of non-digestible fat.

8.) The food of claim 5 wherein said food is a traditional snack.

9.) The food of claim 8 wherein said food is a cracker, filled cracker, potato crisp, extruded snack or filled extruded snack.

10.) The food of claim 5 wherein said food is ready-to-eat.

11.) The composition of claim 1 wherein said composition is a food comprising, on a 30 gram basis:
a.) at least about 0.5 grams of beta-glucan soluble fiber; and
b.) at least about 1 gram of non-digestible fat.

12.) The food of claim 11 wherein said food comprises at least about 0.75 grams of beta-glucan soluble fiber.

13.) The food of claim 11 wherein said food comprises:
a.) from about 0.5 grams to about 7.5 grams of beta-glucan soluble fiber; and
b.) from about 1 gram to about 16.0 grams of non-digestible fat.

14.) The food of claim 11 wherein said food is a traditional snack.

15.) The food of claim 14 wherein said food is a cracker, filled cracker, potato crisp, extruded snack or filled extruded snack.

16.) The food of claim 11 wherein said food is ready-to-eat.

17.) A method for reducing blood cholesterol controlling postprandial blood glucose and insulin levels, in a patient in need of such treatment, comprising administering to said patient:
a.) beta-glucan soluble fiber or a source of beta-glucan soluble fiber; and
b.) a non-digestible fat or a source of non-digestible fat; or
c.) mixtures of (a) and (b); said method comprising oral ingestion, by said patient, of a sufficient amount of component (a) to result in the ingestion of at least about 1.5 g of beta-glucan soluble fiber per day and a sufficient amount of component (b) to result in the ingestion of at least about 10 g of non-digestible fat per day.

18.) A method according to claim 17 which comprises chronic ingestion.

19.) A method according to claim 17 wherein ingestion occurs at two or more regularly-spaced intervals throughout the day.

20.) The method of claim 17 comprising oral ingestion of a sufficient amount of component (a) to result in the ingestion of from about 1.5 g to about 15 g of beta-glucan soluble fiber per day and a sufficient amount of component (b) to result in the ingestion of from about 10 g to about 40 g of non-digestible fat per day.

21.) A method according to claim 20 which comprises chronic ingestion.

22.) A method according to claim 20 wherein ingestion occurs at two or more regularly-spaced intervals throughout the day.

23.) A sheetable dough comprising a sufficient amount of beta-glucan soluble fiber or source of beta-glucan soluble fiber to provide said dough with a beta-glucan soluble fiber level of at least about 1% by weight.

24.) The dough of claim 23 wherein said dough has a beta-glucan soluble fiber level of at least about 2.0% by weight.

25.) The dough of claim 23 wherein said dough has a beta-glucan soluble fiber level from about 1% to about 10% by weight.

26.) The dough of claim 23 comprising a carbohydrate source in addition to said beta-glucan soluble fiber and a fat source.

27.) A food made from the sheetable dough of claim 23.

28.) A traditional snack food comprising, on a 30 gram basis, at least about 0.5 grams of beta-glucan soluble fiber.
